(19) United States
(12) Patent Application Publication
(10) Pub. No.: US 2022/0103926 A1
(43) Pub. Date: Mar. 31, 2022

(54) METHOD OF AUTOMATICALLY CHANGING POWER ON/OFF STATE OF ELECTRONIC HEADPHONES OR ELECTRONIC HEARING PROTECTION EARMUFFS WHEN PLACED ON OR REMOVED FROM THE HEAD OF THE USER

(71) Applicant: Logan Andrew Warren, Seattle, WA (US)

(72) Inventor: Logan Andrew Warren, Seattle, WA (US)

(21) Appl. No.: 17/485,616

(22) Filed: Sep. 27, 2021

Related U.S. Application Data
(60) Provisional application No. 63/084,871, filed on Sep. 29, 2020.

(51) Int. Cl. H04R 1/10; H04R 1/02; H04R 5/033; H04R 5/04

(52) U.S. Cl. CPC H04R 1/1041; H04R 1/028; H04R 5/04; H04R 1/1008; H04R 5/0335

(57) ABSTRACT

An electronic headphone set including a pair of ear cups with a head band interconnecting the ear cups. At least one of the ear cups is movable between a usable position and an unusable position, and electronic circuitry is operably connected to the ear cups. A switch operably interconnects at least one ear cup to the head band and is operable to enable the electronic circuitry when in the usable position and to disable the electronic circuitry when in the unusable position.

### CROSS-REFERENCE TO RELATED APPLICATIONS

This application claims the benefit of U.S. Provisional Patent Application No. 63/084,871, filed on Sep. 29, 2020, entitled “METHOD OF AUTOMATICALLY CHANGING POWER ON/OFF STATE OF ELECTRONIC HEADPHONES OR ELECTRONIC HEARING PROTECTION EARMUFFS WHEN PLACED ON OR REMOVED FROM THE HEAD OF THE USER”, which is hereby incorporated by reference in its entirety for all that is taught and disclosed therein.

### FIELD OF THE INVENTION

The present invention relates to electronic headphones and electronic hearing protectors.

### BACKGROUND AND SUMMARY

Electronic headphones and electronic hearing protectors (both hereinafter referred to as headphones) frequently suffer from several problems.

Problem 1. Electronic headphones must be manually powered on before use. There are no mainstream headphones which have an auto-on/off feature that powers the device on and off when placed on and removed from the head. In the case of wireless headphones, this adds an extra step before the user can use the headphones, frequently requiring the user to cycle through power on/off and wireless pairing modes before the device can be properly powered on. In the case of most electronic hearing protectors, the user must power on the device and select the appropriate volume using the same dial, a process which requires the use of the hands, and may take a significant amount of time to accomplish as the user tries to determine whether or not the power is on, while selecting an appropriate volume.

Problem 2. Electronic headphones must be manually powered off when removed from the head. If the user forgets to power the headphones off, the headphones will continue to draw power and drain batteries or waste electricity. This is especially problematic for battery powered headphones, as the next time the headphones are used, they will be dead. Total battery depletion will also damage the battery or cause battery leakage within the headphones.

Problem 3. In time-critical situations, powering electronic headphones and electronic hearing protectors on and off can consume valuable time. Law enforcement, military, fire, EMT, and civilian persons responding to emergency situations requiring headphones (such as shootings, fires, loud venues, home defense, and emergency personnel running lights and sirens en route to an emergency) may be better served by headphones with an automatic power on/off function.

Past efforts and related technologies for providing automatic power on/off features in headphones:

U.S. Pat. No. 3,862,379 utilizes a spring-operated switch contained in the on-ear headphone, and is activated by exerting pressure on the user's ear.

U.S. Pat. No. 4,677,678 utilizes a complex, bulky, and impractical mechanism on the headband and ear cup of the device.

U.S. Pat. No. 5,144,678 utilizes a microswitch located inside the earcup padding of the headphones (Note: this patent does in FIG. 7 depict an exposed external sliding switch actuated by the arm of the headphones.)

The above disadvantages are addressed by an electronic headphone set including a pair of ear cups with a head band interconnecting the ear cups. At least one of the ear cups is movable between a usable position and an unusable position, and electronic circuitry is operably connected to the ear cups. A switch operably interconnects at least one ear cup to the head band and is operable to enable the electronic circuitry when in the usable position and to disable the electronic circuitry when in the unusable position.

### BRIEF DESCRIPTION OF THE DRAWINGS

FIG. 1 depicts the electronic headphones in the “power off position”. This position will occur when the headphones are not on the head of the wearer. This figure has been drawn to be representative of one possible construction method and does not exclude other hinge/mounting methods that may be utilized. Parts 2-6 may be inferred to exist on the other side of the headphones. If parts 4-6 exist on the other side of the headphones, they may be wired in a manner which requires both ear cups to be in the “power on position” depicted in FIG. 2, to avoid inadvertent activation if one ear cup is unintentionally placed in the “power on position”.

This headphone assembly includes head band 1, generally comprised of an elastic or springy material that allows it to clamp onto the wearer's head when placed on the head.

Ear cup 2 is generally constructed of plastic or metal material, and contains the inner workings of the headphone, which are outside the scope of this provisional patent application. Generally, the headphones will include a speaker, amplification/audio circuitry, noise cancellation microphones and supporting circuitry, a battery, sound insulation material, and other components which may be located around example location 6. Ear cup 2 may also contain a motion sensor and corresponding circuitry designed to sense motion and control the flow of power from the power source.

Ear pad 3 is generally constructed of foam, rubber, silicone, or other material deigned to support the headphones over the ears of the wearer, providing comfort, sound isolation, and supporting the device on the head.

Connection point 4 is a hinge or pivot which allows the angle of the headphones to adjust to the wearer, and can provide telescoping, hinging, and angle adjustment features as needed to suit the specific model or application of the headphones. (Note: the telescoping, hinging, and angle adjustment features of this hinge component are outside the scope of this patent and may be implemented by the manufacturer as desired.)

Pivot arm 5 connects head band 1 to connection point 4. Pivot arm 5 may contain a magnet corresponding to a magnetic proximity sensor or Hall Effect sensor located on or about Location 6. Pivot arm 5 may alternatively be constructed with an interface designed to interact with a microswitch, button, or sensor located on or about Location 6.

Location 6 contains a device designed to interact with pivot arm 5 and provide power on/off features when the headphones are placed on the head or removed from the head. If pivot arm 5 contains a magnet, location 6 may contain a corresponding magnetic proximity or Hall Effect sensor used to control the flow of power from the power source. Alternatively, location 6 may contain a microswitch, button, or sensor designed to interact with pivot arm 5 when pivot arm 5 is moved by the wearer placing the headphones on the head or removing the headphones from the head.

Alternatively, a motion sensor and corresponding circuitry designed to sense motion and control the flow of power from the power source may be utilized to power the device off when motion is not sensed for a period of time.

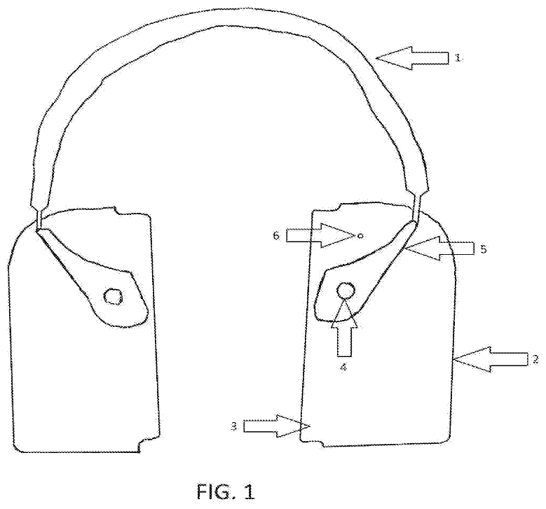

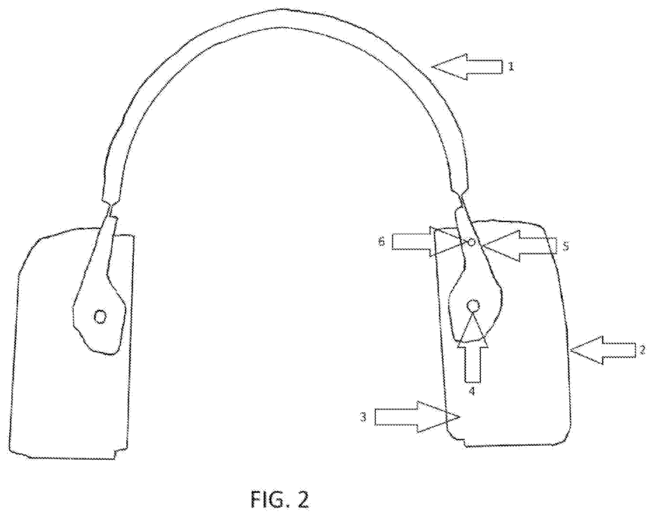

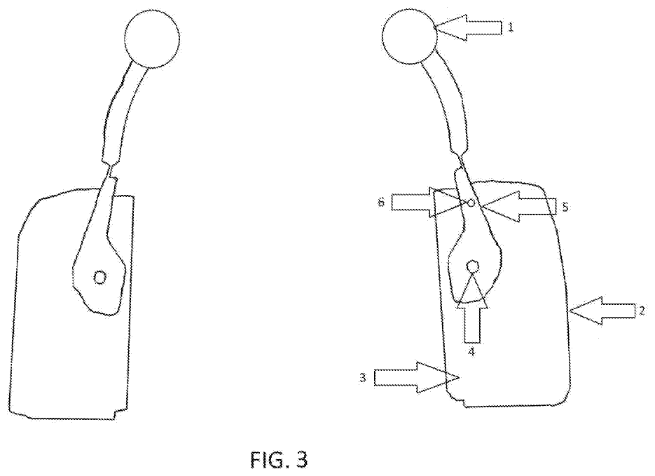

Note that in FIG. 1 “power off position” the pivot arm 5 is out of line with location 6. In this position, the magnetic proximity sensor, Hall Effect Sensor, microswitch, button, or similar sensor will interrupt the flow of electricity from the power source.

FIG. 2 depicts the electronic headphones in the “power on” position. This position will occur when the headphones are placed on the head of the wearer. This figure has been drawn to be representative of one possible construction method and does not exclude other hinge/mounting methods that may be utilized. Parts 2-6 may be inferred to exist on the other side of the headphones. Parts 2-6 may be inferred to exist on the other side of the headphones. If parts 4-6 exist on the other side of the headphones, they may be wired in a manner which requires both ear cups to be in the “power on position” depicted in FIG. 2, to avoid inadvertent activation if one ear cup is unintentionally placed in the “power on position”.

This headphone assembly includes head band 1, generally comprised of an elastic or springy material that allows it to clamp onto the wearer's head when placed on the head.

Ear cup 2 is generally constructed of plastic or metal material, and contains the inner workings of the headphone, which are outside the scope of this provisional patent application. Generally, the headphones will include a speaker, amplification/audio circuitry, noise cancellation microphones and supporting circuitry, a battery, sound insulation material, and other components which may be located around example location 6. Ear cup 2 may also contain a motion sensor and corresponding circuitry designed to sense motion and control the flow of power from the power source.

Ear pad 3 is generally constructed of foam, rubber, silicone, or other material deigned to support the headphones over the ears of the wearer, providing comfort, sound isolation, and supporting the device on the head.

Connection point 4 is a hinge or pivot which allows the angle of the headphones to adjust to the wearer, and can provide telescoping, hinging, and angle adjustment features as needed to suit the specific model or application of the headphones. (Note: the telescoping, hinging, and angle adjustment features of this hinge component are outside the scope of this patent and may be implemented by the manufacturer as desired.)

Pivot arm 5 connects head band 1 to connection point 4. Pivot arm 5 may contain a magnet corresponding to a magnetic proximity sensor or Hall Effect sensor located on or about Location 6. Pivot arm 5 may alternatively be constructed with an interface designed to interact with a microswitch, button, or sensor located on or about Location 6.

Location 6 contains a device designed to interact with pivot arm 5 and provide power on/off features when the headphones are placed on the head or removed from the head. If pivot arm 5 contains a magnet, location 6 may contain a corresponding magnetic proximity or Hall Effect sensor used to control the flow of power from the power source. Alternatively, location 6 may contain a microswitch, button, or sensor designed to interact with pivot arm 5 when pivot arm 5 is moved by the wearer placing the headphones on the head or removing the headphones from the head.

Alternatively, a motion sensor and corresponding circuitry designed to sense motion and control the flow of power from the power source may be utilized to power the device off when motion is not sensed for a period of time.

Note that in FIG. 2 “power on position” the pivot arm 5 is in line with location 6. In this position, the magnetic proximity sensor, Hall Effect Sensor, microswitch, button, or similar sensor will allow the flow of electricity from the power source.

FIG. 3 depicts a possible application for hard hat mounting of electronic hearing protectors with an automatic power on/off function. Hard hat mounted hearing protection is often used in construction or factory environments where hearing protection cannot have an over-head head band due to interference from the hard hat.

Hard hat mounting point 1 is a generic depiction, and utilizes the standard hard hat accessory mounts as found on most hard hats. This standard system is comprised of a standard slot formed into the hard hat, which can mount accessories such as hearing protection headphones, flashlights, or other accessories. The accessories for this mounting system are generally comprised of a tongue or clip which interfaces with this slot and secures the accessory to the hard hat.

Commonly found on hard hat accessory mounts, mounting point 1 will commonly feature a mechanism which allows the headphones to ratcheted away from the wearer's ear to a distance of approximately 1 inch, and allows the headphones to be rotated about the axis at mounting point 1, and swung forwards or backwards to a locked position approximately parallel with the hard hat.

The electronic headphone automatic power on/off features described in FIG. 1 and FIG. 2 may be integrated as depicted in FIG. 1 and FIG. 2.

Alternatively, the electronic headphone automatic power on/off features described in FIG. 1 and FIG. 2 may be integrated into mounting point 1.

To provide automatic power on/off features, a magnet, magnetic proximity sensor, Hall Effect sensor, microswitch, relay, button, or other sensor may be integrated into mounting point 1. When the headphones are placed on the wearer's ears, the magnet, magnetic proximity sensor, Hall Effect sensor, microswitch, relay, button, or other sensor will connect the power source to the load. Pulling the headphones away from the ears and/or rotating them into position parallel with the hard hat will disengage the magnet, magnetic proximity sensor, Hall Effect sensor, microswitch, relay, button, or other sensor, disconnecting the power source from the load.

### DETAILED DESCRIPTION OF A PREFERRED EMBODIMENT

The assembly described in this document seeks to provide automatic power on/off functionality for headphones, providing power to the headphones when placed on the ears or head of the user, and turning off power to the headphones when removed from the ears or head of the user. These automatic power on/off methods may be integrated inside the headphones, retaining any dust resistant and water-resistant functionality of the headphones.

My idea is unique in using magnetic proximity sensors, motion sensors, or a sealed button system which will provide water and dust resistance.

Description of alternative headphone activation methods:

Microswitch. A microswitch may be located around location 5, with a physical mechanical interface designed around location 6. When the headphones are placed on the head, the microswitch and physical mechanical interface will be brought into alignment, and the physical mechanical interface will depress or slide the microswitch, powering the device on and off when placed on or removed from the head.

Microcontroller and Sensor Activation. A variety of sensors, paired with a microcontroller, may be used to control activation. A simple microcontroller, paired with a sensor, could offer approximately 1 month of battery life and provide auto power on/off functionality. A microcontroller paired with motion, infrared, position, accelerometer, Hall Effect, skin conductivity, or other sensors may be used to detect when the headphones are on the wearer's head and power them on or off. Motion sensors and a microcontroller can activate the circuit when motion is detected. Infrared sensors and a microcontroller can activate the circuit when heat from the wearer's head is detected, or when an IR beam is detected. Position sensors can sense the vertical orientation of the headphones and power the circuit on. Accelerometers can be used to sense motion and position and activate the circuit. Skin conductivity sensors can be used to detect when the device is placed on the head. Generally, these sensors and microcontrollers have a passive power draw too great for long term standby mode, and the motion generated by transportation or carrying the device will likely cause unintended activation, but these methods of activation are mentioned as alternatives in case a use case arises.

Bare Metal Contact Activation. Bare metal contacts may be utilized on or about locations 5 and 6, or on rotary hinge 4. When the headset is placed on the head, these contacts will come into alignment, contacting each other and allowing the flow of electricity to power the circuit.

Dual Sensor Activation. The aforementioned activation methods may be implemented on one side of the headset only, or on both sides. Implementing the activation methods on both sides of the headphones will ensure that one side of the headphones accidentally coming into alignment will not result in activation of the headphones.

I claim:

1. An electronic headphone set including:
a pair of ear cups;
a head band interconnecting the ear cups;
at least one of the ear cups movable between a usable position and an unusable position;
electronic circuitry operably connected to the ear cups;
a switch operably interconnecting at least one ear cup to the head band and operable to enable the electronic circuitry when in the usable position and to disable the electronic circuitry when in the unusable position.
